(19) United States
(12) Patent Application Publication
(10) Pub. No.: US 2004/0158880 A1
(43) Pub. Date: Aug. 12, 2004

(54) Suppression of endogenous immunoglobulin expression in transgenic non-human animals expressing humanized or human antibodies

(76) Inventors: Roland Buelow, Palo Alto, CA (US); Wim van Schooten, Sunnyvale, CA (US)

Correspondence Address: HELLER EHRMAN WHITE & MCAULIFFE LLP, 275 MIDDLEFIELD ROAD, MENLO PARK, CA, 94025-3506, US

(21) Appl. No.: 10/774,208

(22) Filed: Feb. 5, 2004

Related U.S. Application Data
(60) Provisional application No. 60/445,393, filed on Feb. 5, 2003.

(51) Int. Cl. A01K 67/027

(52) U.S. Cl. 800/6; 800/14; 800/19

(57) ABSTRACT

The invention provides a novel approach for suppression of endogenous antibody expression in non-human transgenic animals genetically engineered to express one or several human or humanized immunoglobulin transloci. Polyclonal or one or several monoclonal antibodies specific for endogenous IgM and/or IgE heavy and/or light chains are administered to the transgenic animals such that B-cells expressing their endogenous immunoglobulin are depleted and consequently expression of endogenous immunoglobulin is suppressed. Alternatively endogenous immunoglobulin expression may be inhibited through the expression of transgenes encoding such antibodies, including antibody fragments. This method allows the dominant expression of transloci coding for humanized or human antibodies in the blood, milk and eggs of transgenic animals.

This application claims under 35 U.S.C. § 19(e) the benefit of the filing date of provisional application Serial No. 60/445,393 filed on Feb. 5, 2003.

### FIELD OF THE INVENTION

This invention relates to production of human and humanized antibodies in non-human animals genetically engineered to express one or several human or humanized immunoglobulin loci. In particular, the present invention relates to a method to suppress expression of endogenous immunoglobulin in such transgenic non-human animals by administration of antibodies specific for the animals' endogenous IgM and/or IgD surface immunoglobulin heavy and/or light chains, which results in the depletion of early B cells expressing such immunoglobulins. Alternatively, the animals' endogenous immunoglobulin expression may be inhibited through the expression of transgenes encoding antibodies specific for the animals' endogenous IgM and/or IgD surface immunoglobulin heavy and/or light chains, and ultimately leading to B-cell depletion. This method allows the dominant expression of human or humanized antibodies, for example in the blood, milk or eggs of the transgenic animals.

### BACKGROUND OF THE INVENTION

The production of transgenic non-human animals expressing human(ized) immunoglobulin transloci has been described in detail in PCT Publication Nos. WO 02/12437 A2, and WO 92/03918, and in U.S. Pat. No. 5,545,807, the entire disclosures of which are hereby expressly incorporated by reference.

The introduction of human immunoglobulin genes into the genome of mice results in expression of a diversified human antibody repertoire in such mice. To ensure the expression of human antibodies rather than mouse antibodies endogenous loci were inactivated by genetic means. This inactivation entailed the introduction of a mutation in the endogenous heavy and light chain loci such that both loci became incapable of coding for functional immunoglobulins. Most of these methods modify the DNA in the joining (J) gene segment region rendering it incapable to align properly with the constant (C) immunoglobulin gene fragment. Alternative methods to suppress expression of endogenous immunoglobulin in transgenic mice were considered but were shown not to be effective.

Relevant Literature

Allotype suppression in rabbits has been described by R. Mage, Cold Spring Harbor Symposia on Quantitative Biology 32:203-210 (1967); M. Harrison et al., J. Immunology 111(5): 1595-1597 (1973); and R. Mage, Current Topics in Microbiol. and Immunol. 63: 131-152(1974).

Allotype suppression in chickens has been described by M. Ratcliffe and J. Ivanyi, Eur. J. Immunol. 9:847-852 (1979); and M. Ratcliffe and J. Ivanyi, Eur. J. Immunol. 11:301-306(1981).

Antibody diversification by gene conversion in chicken and rabbit has been described by Bucchini et al., Nature 326: 409-11 (1987); Knight et al., Advances in Immunology 56: 179-218 (1994); Langman et al., Res Immunol 144: 422-46 (1993). The generation of mice expressing human-mouse chimeric antibodies has been described by Pluschke et al., Journal of Immunological Methods 215: 27-37 (1998). The generation of mice expressing human-mouse chimeric antibodies with mouse derived membrane and cytoplamic tails has been described by Zou et al., Science 262: 1271-1274 (1993); and Zou et al., Curr Biol., 4: 1099-1103 (1994). The generation of mice expressing human immunoglobulin polypeptides has been described by Bruggemann et al., Curr. Opin. Biotechnol., 8(4): 455-8 (1997); Lonberg et al., Int. Rev. Immunol. 13(1):65-93 (1995); and Neuberger et al., Nature 338: 350-2 (1989). Generation of transgenic mice using a BAC clone has been described by Yang et al., Nat. Biotechnol. 15: 859-65 (1997).

The generation of transgenic rabbits has been described by Fan, J. et al., Pathol Int. 49: 583-94 1(1999); and Brem et al., Mol. Reprod. Dev. 44: 56-62 (1996). Nuclear transfer cloning of rabbits has been described by Stice et al., Biology of Reproduction 39: 657-664 (1988). Rabbits with impaired immunoglobulin expression have been described by Chen et al., J. Immunol. 150:2783-2793 (1993); and Lamoyi E, and Mage R G., J. Exp. Med. 162:1149-1160(1985).

The production of transgenic chicken has been described by Etches et al., Methods in Molecular Biology 62: 433-450 (1997); and Pain et al., Cells Tissues Organs 165(3-4): 212-9 (1999). A gamma-globulinemic chicken has been described by Frommel et al., J. Immunol. 105(1): 1-6 (1970); and Benedict et al., Adv. Exp. Med. Biol. 88(2): 197-205 (1977).

The cloning of animals from cells has been described by T. Wakayama et al., Nature 394:369-374 (1998); J. B. Cibelli et al., Science 280:1256-1258 (1998); J. B. Cibelli et al., Nature Biotechnology 16:642-646 (1998); A. E. Schnieke et al., Science 278: 2130-2133 (1997); and K. H. Campbell et al., Nature 380: 64-66 (1996).

Production of antibodies from transgenic animals is described in U.S. Pat. Nos. 5,814,318; 5,545,807 and 5,570,429. Homologous recombination for chimeric mammalian hosts is exemplified in U.S. Pat. No. 5,416,260. A method for introducing DNA into an embryo is described in U.S. Pat. No. 5,567,607. Maintenance and expansion of embryonic stem cells is described in U.S. Pat. No. 5,453,357.

The production of humanized and human antibodies in transgenic non-human animals is described in PCT Publication No. WO 02/12437, published on Feb. 14, 2002, the disclosure of which is hereby expressly incorporated by reference in its entirety. WO 02/12437 describes genetically engineered non-human animals containing one or more humanized immunoglobulin loci which are capable of undergoing gene rearrangement and gene conversion in transgenic non-human animals, including animals in which antibody diversity is primarily generated by gene conversion to produce diversified humanized antibodies. The humanized antibodies obtained have minimal immunogenicity to humans and are appropriate for use in the therapeutic treatment of human subjects. It further describes novel nucleotide sequences from the 5′ and 3′ flanking regions of immunoglobulin heavy chain constant region segments of various non-human mammalians, such as chickens, cows, sheep, and rabbits. Recombinant vectors in which human immunoglobulin heavy chain gene segments are flanked by sequences homologous to such 5′ and 3′ sequences are shown to be useful for replacing an immunoglobulin heavy chain gene segment of a non-human animal with the corresponding human immunoglobulin heavy chain gene segment.

### SUMMARY OF THE INVENTION

This invention relates to production of humanized antibodies in non-human animals genetically engineered to express one or several humanized immunoglobulin transloci. More particularly, the present invention relates to a method for suppressing the endogenous immunoglobulin loci in transgenic animals containing one or several human(ized) immunoglobulin transloci. The human or humanized transloci are capable of undergoing gene rearrangement and mutational processes in the transgenic animals to produce a diversified human(ized) antibody repertoire substantially in the absence of endogenous immunoglobulin production.

In one aspect, the invention concerns a method of producing humanized or human antibodies comprising:

(a) treating a transgenic non-human animal in which rearrangement of immunoglobulin genes substantially stops early in life, engineered to express one or more humanized or human immunoglobulin loci, with at least one antibody specific for the endogenous surface IgM and/or IgD heavy and/or light chains produced by early B cells of the animal, whereby early B cells expressing the surface IgM and/or IgD heavy and/or light chains are depleted, and

(b) expressing the humanized or human immunoglobulin loci in the animal.

In another aspect, the invention concerns a method for the suppression of endogenous in immunoglobulin expression in a non-human animal comprising expressing in the animal one or more transgenes encoding one or more antibodies specific for the endogenous surface IgM and/or IgD heavy and/or light chains produced by early B cells of such animal.

In yet another aspect, the invention concerns a method for producing a non-human, non-rodent transgenic animal in which endogenous immunoglobulin production is suppressed, comprising treating the non-human, non-rodent transgenic animal with at least one antibody specific for the endogenous surface IgM and/or IgD heavy and/or light chains produced by early B cells of the animal.

In a further aspect, the invention concerns a method for producing a non-human transgenic animal in which endogenous immunoglobulin production is suppressed, comprising expressing in the non-human transgenic animal one or more transgenes encoding one or more antibodies specific for the endogenous surface IgM and/or IgD heavy and/or light chains produced by early B cells of the animal.

In a still further aspect, the invention concerns a non-human transgenic animal dominantly expressing human or humanized antibodies, wherein the animal (1) uses primarily gene conversion and/or other mutational processes to diversify the primary antibody repertoire; (2) expresses a transgene comprising a human immunoglobulin gene or an immunoglobulin gene of the animal modified to express at least part of a human immunoglobulin molecule, and (3) expresses a transgene coding an antibody specific for the endogenous surface IgM and/or IgD heavy and/or light chains produced by early B cells of the animal.

In all aspects, preferred non-human animals are non-rodent animals in which rearrangement of immunoglobulin genes (V, D, and J) substantially stops early in life, and/or which use gene conversion and/or other mutational processes to diversify the antibody repertoire. A preferred group of non-human animals includes rabbits, birds (e.g. chicken, turkey, etc.), cows, pigs, sheep, goats and horses. Another preferred group of non-human animals include rabbits, birds, cows and pigs.

The antibodies used to suppress endogenous antibody production may be polyclonal antibody preparations or monoclonal antibodies, including antibody fragments and immunotoxins.

In all embodiments, analogous methods may be performed by toxins specific for the B cells of the non-human transgenic animals the endogenous immunoglobulin production of which is to be suppressed.

### DETAILED DESCRIPTION OF THE INVENTION

Definitions

“Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. The term “antibody” is used herein in the broadest sense and specifically covers, without limitation, monoclonal antibodies (including full length monoclonal antibodies), polyclonal antibodies, multispecific antibodies (e.g., bispecific antibodies), and antibody fragments so long as they exhibit the desired specificity.

The term “monoclonal antibody” is used to refer to an antibody molecule synthesized by a single clone of B cells.

The term “polyclonal antibody” is used to refer to a population of antibody molecules synthesized by a population of B cells.

“Native antibodies” and “native immunoglobulins” are usually heterotetrameric glycoproteins of about 150,000 daltons, composed of two identical light (L) chains and two identical heavy (H) chains. Each light chain is linked to a heavy chain by covalent disulfide bond(s), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains (Clothia et al., J. Mol. Biol. 186:651 (1985); Novotny and Haber, Proc. Natl. Acad. Sci. U.S.A. 82:4592 (1985)).

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, connected by three CDRs. The CDRs in each chain are held together in close proximity by the FR regions and, with the CDRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in binding an antibody to an antigen, but exhibit various effector functions, such as participation of the antibody in antibody-dependent cellular toxicity.

The terms “humanized antibody” and “humanized immunoglobulin,” as used herein, mean an immunoglobulin molecule comprising at least a portion of a human immunoglobulin polypeptide sequence (or a polypeptide sequence encoded by a human immunoglobulin gene segment). The humanized immunoglobulin molecules of the present invention can be isolated from a transgenic non-human animal engineered to produce humanized immunoglobulin molecules. Such humanized immunoglobulin molecules are less immunogenic to primates, especially humans, relative to non-humanized immunoglobulin molecules prepared from the animal or prepared from cells derived from the animal. The terms “human antibody” and “human immunoglobulin” are used herein to refer to an immunoglobulin molecule comprising fully human sequences.

The term “non-human animal” as used herein includes, but is not limited to mammals, and includes, for example, non-human primates, rodents (e.g. mice and rats), non-human, non-rodent animals, such as, for example, rabbits, pigs, birds (e.g., chickens, turkeys, ducks, geese and the like), sheep, goats, cows, pigs, and horses. Preferred non-human animals are those where gene rearrangement stops early in life, such as, for example, rabbits, birds (e.g., chickens, turkeys, ducks, geese, and the like), sheep, goats, cows, pigs, and horses. Another preferred group includes animals which create antibody diversity substantially by gene conversion and/or other mutational processes, such as somatic hypermutation to generate antibody diversity, e.g., rabbits, birds (e.g., chickens, turkeys, ducks, geese and the like), cows and pigs. Particularly preferred non-human animals are rabbits and chickens.

The term “non-primate animal” as used herein includes, but is not limited to, mammals other than primates, including but not limited to the mammals specifically listed above.

The phrase “animals which rely substantially on gene conversion and/or other mutational processes to create primary antibody repertoires,” and its grammatical equivalents, are used to refer to animals in which the predominant mechanism of antibody diversification is gene conversion and/or a mutational process, such as, e.g. (somatic) hypermutation as opposed to gene rearrangement.

The term “Ig gene segment” as used herein refers to segments of DNA encoding various portions of an Ig molecule, which are present in the germline of animals and humans, and which are brought together in B cells to form rearranged Ig genes. Thus, Ig gene segments as used herein include V gene segments, D gene segments, J gene segments and C region gene segments.

The term “human Ig gene segment” as used herein includes both naturally occurring sequences of a human Ig gene segment, degenerate forms of naturally occurring sequences of a human Ig gene segment, as well as synthetic sequences that encode a polypeptide sequence substantially identical to the polypeptide encoded by a naturally occurring sequence of a human Ig gene segment. In this context, by “substantially” is meant that the degree of amino acid sequence identity is at least about 85%-95%. In a particular embodiment, the human Ig gene segment renders the immunoglobulin molecule non-immunogenic in humans.

The term “diabodies” refers to small antibody fragments with two antigen-binding sites, which fragments comprise a heavy chain variable domain (V_{H}) connected to a light chain variable domain (V_{L}) in the same polypeptide chain (V_{H}-V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout this application refers to the antibodies described in Zapata et al., Protein Eng. 8(10):1057-1062 (1995), and the like. Briefly, these antibodies comprise a pair of tandem Fd segments (V_{H}-C_{HI}-V_{H}-C_{HI}) which form a pair of antigen binding regions. Linear antibodies can be bispecific or monospecific.

The terms “antibody diversity” and “antibody repertoire” are used interchangeably, and refer to the total of all antibody specificities that an organism is capable of expressing.

The term “antibodies specific for endogenous surface IgM and/or IgD heavy and/or light chain” as used herein refers to polyclonal antibody preparations and monoclonal antibodies, which specifically bind to an IgM and/or IgD molecule produced by a non-human animal relative to binding to a corresponding human immunoglobulin. In other words, such antibodies bind to a region within the sequence of an endogenous IgM and/or IgD heavy or light chain which includes at least one unique epitope relative to a corresponding human sequence. Specific binding means that an antibody binds to the target endogenous non-human immunoglobulin without exhibiting any significant binding to a human immunoglobulin.

### DETAILED DESCRIPTION

The present invention provides non-genetic methods for the suppression of the endogenous immunoglobulin production in non-human animals, to render the animals more suitable for the expression of human(ized) immunoglobulin(s). In a particular embodiment, the invention provides method for the suppression of the endogenous immunoglobulin production in non-human, non-rodent animals.

According to the present invention, endogenous immunoglobulin production is suppressed by treating a target non-human animal with a polyclonal antibody preparation or one or more monoclonal antibodies that specifically bind a cell surface IgM and/or IgD heavy and/or light chains produced by B cells of such animal. B cells develop from hematopoietic stem cells. Prior to antigen exposure, B cells undergo a series of maturation steps the end product of which is a mature B cell, which expresses a unique membrane-associated IgM and often IgD on its cell surface along with other cell surface signaling molecules. While in humans antibody diversification by gene rearrangement occurs throughout life, in other animals the diversification of antibody repertoire stops early in life, typically within the first month of life. Binding of mono- or polyclonal antibodies to IgM and/or IgD heavy and/or light chains expressed on the surface of mature B cells (prior to isotype switching) results in the death of such B cells. In animals where rearrangement of immunoglobulin genes stops early in life, killing all or most of B cells produced during this limited period of time effectively results in lasting or permanent arrest of endogenous immunoglobulin production. In transgenic animals, which contain one or several human or humanized immunoglobulin transloci, this enables the production of human or humanized immunoglobulin, in the absence of endogenous immunoglobulin production of the animal. In this way, the expression of the endogenous immunoglobulin(s) can be effectively suppressed in animals where gene rearrangement stops early in live. Examples of such animals are, without limitation, rabbits, birds (e.g. chickens), sheep, goats, cattle, swine and horses. Injection of polyclonal or one or several monoclonal antibodies specific for the animals' endogenous IgM and/or IgD immunoglobulin(s) heavy and/or light chains into either the embryo itself or indirectly via the mother has been shown to effectively suppress the targeted locus. Examples for such antibodies are antibodies specifically binding the animals' light and/or heavy chain immunoglobulin molecules. Alternatively, antibodies specific for endogenous immunoglobulin(s) are injected into the animals shortly after birth or hatching. Both methods have been shown to very effectively suppress expression of the targeted immunoglobulin locus in chickens and rabbits which express heavy or light chain immunoglobulin alleles having different allotypes. Allotypes are formed by combinations of amino acids in the V(D)J regions of the immunoglobulin heavy and light chains.

For example, injection of allotype-specific antibodies into 13-day old chicken embryos results in effective suppression of the endogenous locus expressing the targeted allotype. Total immunoglobulin levels are normal in these animals but 99% of the immunoglobulins express the non-targeted allotype. This suppression can last for more than 12 months.

In a similar manner, suppression of one allele can be achieved in rabbits by injection of allotype-specific antibodies into the mother and/or into newly born rabbits during the first 2 weeks of life. However, suppression of both alleles in heterozygous animals has been shown to be difficult and only transient suppression can be achieved; immunoglobulin titers return to normal levels within weeks after injection of allotypes-specific antibodies. Likewise suppression of allotypes in homozygous animals has been shown to result in only a temporary reduction of immunoglobulin concentrations in the blood.

Two basic antibody maturation phenomena contribute to effective allotype suppression in rabbits and chickens, namely: (i) only very few V genes are expressed which are subsequently diversified by mutational processes, (ii) recombination of V, D, and J genes stops in the first month after birth in rabbits and at hatching in chickens, respectively. For these reasons, removal of subpopulations of B-cells expressing one allotype during embryonic or early life will result in lifelong suppression of immunoglobulin production of that allotype and dominant production of the other allotype. Analogously, suppression of both endogenous immunoglobulin loci in transgenic animals results in dominant expression of the immunoglobulin encoded by the transgene.

The invention provides a novel approach for the suppression of endogenous immunoglobulin production in transgenic non-human animals capable of producing human(ized) immunoglobulins. More specifically, polyclonal or one or several monoclonal antibodies specific for B-cells expressing the animals' endogenous immunoglobulin heavy and/or light chains are administered to transgenic non-human animals.

In a preferred embodiment, endogenous immunoglobulin production is suppressed by the administration of polyclonal or one or several monoclonal antibodies specific for a B cell marker expressing the animal's endogenous immunoglobulin. The B cell marker must not be present or sufficiently different on B cells expressing human(ized) immunoglobulin. Typically, the B cell marker is an IgM and/or an IgD heavy and/or light chain, which is of a different allotype than the corresponding human immunoglobulin.

As noted before, the treatment may be performed with polyclonal antibody preparations or one or more monoclonal antibodies, specifically binding to an IgM and/or IgD heavy and/or light chains molecule expressed on the surface of B cells of the animal.

Allotype-specific antibodies are known in the art and can be readily made by techniques known in the art for making mono- or polyclonal antibodies. For example, anti-IgM1-b- and anti-IgM1-a-specific monoclonal antibodies for allotype suppression in the chicken have been described by Ratcliffe, Cell. Immunol. 83(1):208-14 (1984); Ratcliffe and Ivanyi, Eur. J. Immunol. 11(4):301-6 (1981); and Ratcliffe and Ivanyi, Eur. J. Immunol. 11(4):296-36 (1981). The first chicken immunoglobulin light (L) chain allotypic specificity (L-1.1) that was present on IgM, 7S Ig, Fab, and L chains was reported by Foppoli and Benedict, J. Immunol. 122(5):1681-5 (1979). Rabbit IgM allotypes have been described, for example, by Gilman-Sachs et al., J. Immunol. 128(1):451-6 (1982). Allotype suppression of IgM in rabbits by antibodies directed towards the CH region has been reported by Gilman-Sachs and Eskinazi, J. Immunol. 119(4):1369-73 (1977). One of skill in the art will understand that these reports are for illustration only. Specific allotypes in other animals and antibodies binding to such specific allotypes are well known in the art, and can be use to practice the present invention.

These specific antibodies may, for example, be given to the transgenic animals during embryonic life by directly injecting the antibodies into the embryo or indirectly by injecting them into the pregnant mother or into the egg-laying hen. As a consequence B-cells expressing the animal's immunoglobulin molecules (endogenous immunoglobulins) are deleted and hence transgenic offspring will predominantly produce human(ized) antibodies in response to immunization with antigens.

In another embodiment, the animal's endogenous immunoglobulin production is suppressed by administering polyclonal or one or several monoclonal antibodies specific for the animal's endogenous surface IgM and/or IgD expressed by early B cells, within the first 2 weeks of life. This treatment deletes B-cells expressing endogenous immunoglobulin molecules. Transgenic animals appropriately treated produce predominantly human(ized) antibodies in response to immunogens.

In another embodiment, B-cells expressing the animals' endogenous immunoglobulin can also be depleted through expression of transgenes encoding one or several monoclonal antibodies specific for the animal's endogenous (IgM and/or IgD heavy and/or light chains) immunoglobulin. Such additional transgenes encoding one or several monoclonal antibodies can be introduced into the animal's genome by methods known in the art, such as by using produces injection or other genetic engineering techniques (nuclear transfer cloning, embryonic stem cells, etc). In such animals expression of one or several monoclonal antibodies or toxins specific for IgM and/or IgD heavy or light chains expressed on the surface of early B-cells results in the depletion of such B-cells without effect on B-cells expressing human(ized) immunoglobulin.

In all embodiments, the antibodies include antibody fragments, multispecific antibodies, diabodies, heteroconjugate antibodies, and immunoconjugates. Thus, the immunoconjugates may comprise an antibody, including antibody fragments, conjugated to a cytotoxic agent such as a chemotherapeutic agent, toxin (e.g. an enzymatically active toxin of bacterial, fungal, plant or animal origin, or fragments thereof), or a radioactive isotope (i.e., a radioconjugate). Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. A variety of radionuclides are also available for the production of radioconjugate antibodies. Examples include .²¹²Bi, . ¹³³I, .¹³¹In, ⁹⁰Y and ¹⁸⁶Re. Conjugates of the monoclonal antibody and cytotoxic moieties can be made using a variety of bifunctional coupling agents such as, for example, SPDP, IT, bifunctional derivatives of imidoesters such a dimethyl adipimidate HCl, active esters such as disuccinimidyl suberate, aldehydes such as glutaraldehyde, bis-azido compounds such as bis (p-azidobenzoyl) hexanediamine, bis-diazonium derivatives such as bis-(p-diazoniumbenzoyl)-ethylenediamine, diisocyanates such as tolylene 2,6-diisocyanate, and bis-active fluorine compounds such as 1,5-difluoro-2,4-dinitrobenzene. The lysing portion of a toxin may be joined to the Fab fragment of the antibodies.

The B-cell specific (anti-IgM and/or anti-IgD) antibodies may exert their activity by a variety of mechanisms, including antibody-dependent cellular cytotoxicity (ADCC), growth inhibitory, cytotoxic, and/or apoptotic activities. Antibodies of different classes and subclasses differ in this respect, and are all included within the scope of the present invention. In general, antibodies of the IgG2a and IgG3 subclass and occasionally IgG1 can mediate ADCC, and antibodies of the IgG3, and IgG2a and IgM subclasses bind and activate serum complement. Complement activation generally requires the binding of at least two IgG molecules in close proximity on the target cell. However, the binding of only one IgM molecule activates serum complement.

As noted before, in an important embodiment, the endogenous immunoglobulin production of transgenic animals engineered to express human or humanized immunoglobulins is suppressed according to the present invention. The transgene(s) encoding human(ized) antibodies contain(s) an Ig locus or a large portion of an Ig locus from the animal, wherein the Ig locus or part thereof has been genetically modified to contain one or several of a human Ig gene segment (e.g., a human Ig V, D, J or C gene segment). Alternatively, the transgene is a human immunoglobulin locus or a large portion thereof. The transgene containing such modified Ig locus or modified portion of an Ig locus, also referred to herein as “a humanized Ig translocus”, is capable of undergoing gene rearrangement in the transgenic non-human animal thereby producing a diversified repertoire of antibodies having at least a portion of a human immunoglobulin polypeptide sequence. Suppression of the endogenous immunoglobulin production results in the dominant expression of the humanized or human Ig translocus. Immunization with antigen leads to the production of humanized or human antibodies against the same antigen in said transgenic animals.

Although preferred embodiments of the present invention are directed to transgenic animals having humanized Ig loci and producing humanized polyclonal antisera, it is to be understood that transgenic animals having primatized Ig loci and primatized polyclonal antisera are also within the spirit of the present invention. Similar to humanized polyclonal antisera compositions, primatized polyclonal antisera compositions likely have a reduced immunogenicity in human individuals.

Once a transgenic non-human animal capable of producing diversified humanized immunoglobulin molecules is made (as further set forth below), humanized immunoglobulins and humanized antibody preparations against an antigen can be readily obtained by immunizing the animal with the antigen. A variety of antigens can be used to immunize a transgenic host animal. Such antigens include, microorganism, e.g. viruses and unicellular organisms (such as bacteria and fungi), alive, attenuated or dead, fragments of the microorganisms, or antigenic molecules isolated from the microorganisms.

Preferred bacterial antigens for use in immunizing an animal include purified antigens from Staphylococcus aureus such as capsular polysaccharides type 5 and 8, recombinant versions of virulence factors such as alpha-toxin, adhesin binding proteins, collagen binding proteins, and fibronectin binding proteins. Preferred bacterial antigens also include an attenuated version of S. aureus, Pseudomonas aeruginosa, enterococcus, enterobacter, and Klebsiella pneumoniae, or culture supernatant from these bacteria cells. Other bacterial antigens which can be used in immunization include purified lipopolysaccharide (LPS), capsular antigens, capsular polysaccharides and/or recombinant versions of the outer membrane proteins, fibronectin binding proteins, endotoxin, and exotoxin from Pseudomonas aeruginosa, enterococcus, enterobacter, and Klebsiella pneumoniae.

Preferred antigens for the generation of antibodies against fungi include attenuated version of fungi or outer membrane proteins thereof, which fungi include, but are not limited to, Candida albicans, Candida parapsilosis, Candida tropicalis, and Cryptococcus neoformans.

Preferred antigens for use in immunization in order to generate antibodies against viruses include the envelop proteins and attenuated versions of viruses which include, but are not limited to respiratory synctial virus (RSV) (particularly the F-Protein), Hepatitis C virus (HCV), Hepatits B virus (HBV), cytomegalovirus (CMV), EBV, and HSV.

Therapeutic antibodies can be generated for the treatment of cancer by immunizing transgenic animals with isolated tumor cells or tumor cell lines; tumor-associated antigens which include, but are not limited to, Her-2-neu antigen (antibodies against which are useful for the treatment of breast cancer); CD19, CD20, CD22 and CD53 antigens (antibodies against which are useful for the treatment of B cell lymphomas), (3) prostate specific membrane antigen (PMSA) (antibodies against which are useful for the treatment of prostate cancer), and 17-1A molecule (antibodies against which are useful for the treatment of colon cancer).

The antigens can be administered to a transgenic host animal in any convenient manner, with or without an adjuvant, and can be administered in accordance with a predetermined schedule.

After immunization, serum or milk from the immunized transgenic animals can be fractionated for the purification of pharmaceutical grade polyclonal antibodies specific for the antigen. In the case of transgenic birds, antibodies can also be made by fractionating egg yolks. A concentrated, purified immunoglobulin fraction may be obtained by chromatography (affinity, ionic exchange, gel filtration, etc.), selective precipitation with salts such as ammonium sulfate, organic solvents such as ethanol, or polymers such as polyethyleneglycol.

The fractionated human(ized) antibodies may be dissolved or diluted in non-toxic, non-pyrogenic media suitable for intravenous administration in humans, for instance, sterile buffered saline.

The antibody preparations used for administration are generally characterized by having immunoglobulin concentrations from 0.1 to 100 mg/ml, more usually from 1 to 10 mg/ml. The antibody preparation may contain immunoglobulins of various isotypes. Alternatively, the antibody preparation may contain antibodies of only one isotype, or a number of selected isotypes.

For making a human or humanized monoclonal antibody, spleen cells are isolated from the immunized transgenic animal whose B-cells expressing the animal's endogenous immunoglobulin have been depleted. Isolated spleen cells are used either in cell fusion with transformed cell lines for the production of hybridomas, or cDNAs encoding antibodies are cloned by standard molecular biology techniques and expressed in transfected cells. The procedures for making monoclonal antibodies are well established in the art. See, e.g., European Patent Application 0 583 980 A1 (“Method For Generating Monoclonal Antibodies From Rabbits”), U.S. Pat. No. 4,977,081 (“Stable Rabbit-Mouse Hybridomas And Secretion Products Thereof”), WO 97/16537 (“Stable Chicken B-cell Line And Method of Use Thereof”), and EP 0 491 057 B1 (“Hybridoma Which Produces Avian Specific Immunoglobulin G”), the disclosures of which are incorporated herein by reference. In vitro production of monoclonal antibodies from cloned cDNA molecules has been described by Andris-Widhopf et al., “Methods for the generation of chicken monoclonal antibody fragments by phage display”, J Immunol Methods 242:159 (2000), and by Burton, D. R., “Phage display”, Immunotechnology 1:87 (1995), the disclosures of which are incorporated herein by reference.

In most instances the antibody preparation consists of unmodified immunoglobulins, i.e., humanized antibodies prepared from the animal without additional modification, e.g., by chemicals or enzymes. Alternatively, the immunoglobulin fraction may be subject to treatment such as enzymatic digestion (e.g. with pepsin, papain, plasmin, glycosidases, nucleases, etc.), heating, etc, and/or further fractionated.

The invention is further illustrated, but by no means limited, by the following examples.

### EXAMPLE 1

### Deletion of B-Cells Expressing Endogenous Immunoglobulin in Transgenic Rabbits By Injection of Rabbit Immunoglobulin Specific Polyclonal Antisera

Transgenic rabbits expressing humanized heavy and light chain immunoglobulin proteins are injected with polyclonal antibodies specific for endogenous immunoglobulin molecules. A polyclonal antibody preparation is produced by immunizing rabbits expressing a particular allotype with a purified Ig protein fraction from rabbits expressing a different allotype together with Freund adjuvant. After collection of serum from these immunized animals specific antibodies are generated by; (i) positively selecting rabbit reactive antibodies from the antiserum using a column having a matrix coupled with rabbit immunoglobulins, and (ii) by removing human(ized) immunoglobulin reactive antibodies by collecting the flow through of a column having a matrix coupled with human(ized) immunoglobulin. The rabbit Ig specific polyclonal antibodies are administered to transgenic rabbits expressing humanized immunoglobulin genes. Antibodies are injected into 1-5 day old newborns at 1-100 mg per kilo bodyweight or into mothers pregnant of transgenic rabbits. B-cells are analyzed 4 weeks after treatment which reveals that B-cells expressing endogenous Ig molecules is 1-10% of normal and 10-100 times lower than B-cells expressing humanized immunoglobulin transgenes. Analysis of immunoglobulin concentrations in the blood shows that endogenous immunoglobulin concentrations are 5-100 fold lower than humanized immunoglobulin concentrations.

### EXAMPLE 2

### Deletion of B-Cells Expressing Endogenous Immunoglobulin in Transgenic Rabbits By Injection of Rabbit Immunoglobulin Specific Polyclonal Antisera Made in Goats

Transgenic rabbits expressing humanized heavy and light chain immunoglobulin proteins are injected with polyclonal antibodies specific for endogenous immunoglobulin molecules. A polyclonal antibody preparation is produced by immunizing goats with a purified IgM protein fraction from rabbits together with Freund adjuvant. After collection of serum from these immunized animals specific antibodies are generated by; (i) positively selecting rabbit reactive antibodies from the antiserum using a column having a matrix coupled with rabbit immunoglobulins, and (ii) by removing human(ized) immunoglobulin reactive antibodies by collecting the flow through of a column having a matrix coupled with human(ized) immunoglobulin. The rabbit Ig specific polyclonal antibodies are administered to transgenic rabbits expressing humanized immunoglobulin genes. Antibodies are injected into 1-5 day old newborns at 1-100 mg per kilo bodyweight or into mothers pregnant of transgenic rabbits. B-cells are analyzed 4 weeks after treatment which reveals that B-cells expressing endogenous Ig molecules is 1-10% of normal and 10-100 times lower than B-cells expressing humanized immunoglobulin transgenes. Analysis of immunoglobulin concentrations in the blood shows that endogenous immunoglobulin concentrations are 5-100 fold lower than humanized immunoglobulin concentrations.

### EXAMPLE 3

### Deletion of B-Cells Expressing Endogenous Immunoglobulin in Transgenic Chickens After Treatment With Polyclonal Antisera Specific For Endogenous Immunoglobulin Made in Chickens

Transgenic chickens expressing humanized heavy and light chain molecules are injected with polyclonal antibodies specific for endogenous immunoglobulin molecules. A polyclonal antibody preparation is produced by immunizing chickens expressing a particular allotype with a purified Ig protein fraction from chickens expressing a different allotype together with Freund adjuvant. After collection of the Ig fraction from immunized chickens specific antibodies are generated by; (i) positively selecting chicken reactive antibodies from the antiserum using a column having a matrix coupled with the immunogen and/or chicken immunoglobulins, and (ii) by removing human(ized) immunoglobulin reactive antibodies by collecting the flow through of a column having a matrix coupled with human(ized) immunoglobulin. The endogenous Ig specific polyclonal antibodies are administered to transgenic chickens expressing humanized immunoglobulin genes. Antibodies are injected into 13-day-old embryos at 0.1-10 mg per embryo or into the egg-laying hen at 1-100 mg per kilo weight. B-cells of the blood are analyzed 4 weeks after hatching which reveals that B-cells expressing endogenous Ig molecules is 1-10% of non-transgenic chickens and 10-100 times lower than B-cells expressing humanized immunoglobulin transgenes. Analysis of immunoglobulin concentrations in the blood shows that endogenous immunoglobulin concentrations are 10-100 fold lower than humanized immunoglobulin concentration.

### EXAMPLE 4

### Deletion of B-Cells Expressing Endogenous Immunoglobulin in Transgenic Chickens After treatment with Polyclonal Antisera Specific For Endogenous Immunoglobulin Made in Rabbits

Transgenic chickens expressing humanized heavy and light chain molecules are injected with polyclonal antibodies specific for endogenous immunoglobulin molecules. A polyclonal antibody preparation is produced by immunizing rabbits with a purified IgM protein fraction from chickens together with Freund adjuvant. After collection of the IgM fraction from immunized rabbits specific antibodies are generated by; (i) positively selecting chicken reactive antibodies from the antiserum using a column having a matrix coupled with the immunogen and/or chicken immunoglobulins, and (ii) by removing human(ized) immunoglobulin reactive antibodies by collecting the flow through of a column having a matrix coupled with human(ized) immunoglobulin. The endogenous Ig specific polyclonal antibodies are administered to transgenic chickens expressing humanized immunoglobulin genes. Antibodies are injected into 13-day-old embryos at 0.1-10 mg per embryo or into the egg-laying hen at 1-100 mg per kilo weight. B-cells of the blood are analyzed 4 weeks after hatching which reveals that B-cells expressing endogenous Ig molecules is 1-10% of non-transgenic chickens and 10-100 times lower than B-cells expressing humanized immunoglobulin transgenes. Analysis of immunoglobulin concentrations in the blood shows that endogenous immunoglobulin concentrations are 10-100 fold lower than humanized immunoglobulin concentration.

### EXAMPLE 5

Deletion of B-Cells Expressing Rabbit Immunoglobulin By Expression of Anti-Rabbit IgM Monoclonal Antibody Encoded by a Transgene

A transgene containing the reverse tetracycline-controlled transactivator (rtTA) under the control of the rabbit albumin promoter is constructed. rtTA activates transcription in the presence of tetracycline. Heavy and light chains of an anti-rabbit monoclonal antibody are cloned into pTRE2 which encodes the tetracycline response element. These transgene are introduced into fertilized rabbit oocytes using standard transgenic techniques. In double transgenic founder animals administration of tetracycline results in anti-rabbit IgM antibody expression in hepatocytes but not in spleen.

Transgenic rabbits containing humanized heavy and light chain immunoglobulin loci are bred with transgenic founder animals containing transgenes encoding rtTA and anti-rabbit IgM. This results in offspring with four transgenes: (i) humanized heavy chain locus, (ii) humanized light chain locus, (iii) rtTA, and (iv) anti-rabbit IgM. Pregnant does are treated with tetracycline for 10 days starting 18 days after fertilization. This results in the expression of anti-IgM antibody in the pregnant doe. Expression of anti-rabbit IgM results in depletion of B-cells expressing rabbit IgM in the doe and embryos. No effect on B cell expressing humanized immunoglobulin is detected. Rabbit antibody expression in live offspring is reduced 10-100 fold compared to offspring from does not treated with tetracycline.

Although the present invention is illustrated with reference to certain embodiments, it is not so limited. Indeed, various modifications of the invention in addition to those shown and described herein will become apparent to those skilled in the art from the foregoing description and fall within the scope of the appended claims.

What is claimed is:

1. A method of producing humanized or human antibodies comprising:
(a) treating a transgenic non-human animal in which rearrangement of immunoglobulin genes substantially stops early in life, engineered to express one or more humanized or human immunoglobulin loci, with at least one antibody specific for the endogenous surface IgM and/or IgD heavy and/or light chains produced by early B cells of said animal, whereby early B cells expressing said surface IgM and/or IgD heavy and/or light chains are depleted, and
(b) expressing said humanized or human immunoglobulin loci in said animal.

2. The method of claim 1 wherein said animal relies substantially on gene conversion and/or other mutational processes to create primary antibody diversity.

3. The method of claim 1 wherein said animal substantially stops antibody diversification by gene rearrangement within the first month of its life.

4. The method of claim 1 wherein said animal is selected from the group consisting of rabbits, birds, cows, pigs, sheep, goats and horses.

5. The method of claim 4 wherein said animal is selected from the group consisting of rabbits, birds, cows and pigs.

6. The method of claim 5 wherein said animal is a transgenic rabbit or a transgenic chicken expressing humanized or human immunoglobulin transgenes.

7. The method of claim 1 wherein said antibody is a polyclonal antibody preparation.

8. The method of claim 7 wherein said polyclonal antibody preparation comprises antibody fragments.

9. The method of claim 7 wherein said polyclonal antibody preparation is injected into said animal during embryonic life or the first weeks of life.

10. The method of claim 7 wherein said polyclonal antibody preparation is injected into said transgenic animal during the first two weeks of its life.

11. The method of claim 7 wherein said polyclonal antibody preparation is injected into said transgenic animal during the first month of its life.

12. The method of claim 1 wherein said antibody is a monoclonal antibody.

13. The method of claim 12 wherein said antibody is an antibody fragment.

14. The method of claim 11 wherein more than one monoclonal antibody is administered.

15. The method of any one of claims 1, 7, 8, 12 and 13, wherein said antibody is conjugated to a toxin to form an immunotoxin.

16. A method for suppressing endogenous immunoglobulin expression in a non-human animal comprising expressing in said animal one or more transgenes encoding one or more antibodies specific for the endogenous surface IgM and/or IgD heavy and/or light chains produced by early B cells of said non-human animal.

17. The method of claim 16 wherein said animal is a transgenic animal expressing a human or humanized antibody.

18. The method of claim 17 wherein said transgenic animal is a non-rodent animal.

19. The method of claim 18 wherein said non-rodent transgenic animal relies substantially on gene conversion and/or other non-templated mutational processes to create primary antibody repertoires.

20. The method of claim 18 wherein said transgenic non-rodent animal substantially stops antibody diversification by gene rearrangement early in life.

21. The method of claim 20 wherein said transgenic non-rodent animal substantially stops antibody diversification by gene rearrangement within the first month of its life.

22. The method of claim 18 wherein said non-rodent transgenic animal is selected from the group consisting of rabbits, birds, cows, pigs, sheep, goats and horses.

23. The method of claim 22 wherein said non-rodent transgenic animal is selected from the group consisting of rabbits, birds, cows and pigs.

24. A method for producing a non-human, non-rodent transgenic animal in which endogenous immunoglobulin production is suppressed, comprising treating said non-human, non-rodent transgenic animal with at least one antibody specific for the endogenous surface IgM and/or IgD heavy and/or light chains produced by early B cells of said animal.

25. A method for producing a non-human transgenic animal in which endogenous immunoglobulin production is suppressed, comprising expressing in said non-human transgenic animal one or more transgenes encoding one or more antibodies specific for the endogenous surface IgM and/or IgD heavy and/or light chains produced by early B cells of said animal.

26. A non-human transgenic animal dominantly expressing human or humanized antibodies, wherein said animal (1) uses primarily gene conversion and/or other mutational processes to diversify the primary antibody repertoire; (2) expresses a transgene comprising a human immunoglobulin gene or an immunoglobulin gene of said animal modified to express at least part of a human immunoglobulin molecule, and (3) expresses a transgene coding for one or several antibodies specific for endogenous surface IgM and/or IgD heavy and/or light chains produced by early B cells of said animal

27. The non-human transgenic animal of claim 26 which expresses one or several transgenes coding for one or several monoclonal antibodies capable of suppressing production of endogenous immunoglobulin μ or λ heavy and/or light chains.

28. The non-human transgenic animal of claim 26 selected from the group consisting of rabbits, birds, sheep, goat, pigs, cows and horses.
